(19) United States
(12) Patent Application Publication
(10) Pub. No.: US 2002/0078966 A1
(43) Pub. Date: Jun. 27, 2002

(54) Action inducing device

(76) Inventor: Selwyn L. Lewis, Thornhill (CA)

Correspondence Address: Kaardal & Associates, PC, Attn: Ivar M. Kaardal, Suite 250, 3500 South First Ave. Circle, Sioux Falls, SD, 57105-5802, US

(21) Appl. No.: 09/747,879

(22) Filed: Dec. 26, 2000

(51) Int. Cl. A61B 19/00

(52) U.S. Cl. 128/897

(57) ABSTRACT

A action inducing device for giving instructions to complete various tasks in a plurality of languages. The action inducing device includes a microprocessor adapted for processing received electronic data and sending electronic audio signals. A plurality of storage medium electronically stores data. Each of the storage mediums has a different set of instructions thereon. Each of the storage medium provides one set of instructions in a plurality of languages. A reading device reads the electronically stored data on the storage media. The reading device is electrically coupled to the microprocessor. A is electrically coupled to the microprocessor. A plurality of actuators is used for selecting one of the languages. The actuators are operationally coupled to the microprocessor.

### BACKGROUND OF THE INVENTION

1. Field of the Invention

The present invention relates to action inducing devices and more particularly pertains to a new action inducing device for giving instructions to complete various tasks in a plurality of languages.

2. Description of the Prior Art

The use of action inducing devices is known in the prior art. More specifically, action inducing devices heretofore devised and utilized are known to consist basically of familiar, expected and obvious structural configurations, notwithstanding the myriad of designs encompassed by the crowded prior art which have been developed for the fulfillment of countless objectives and requirements.

Known prior art includes U.S. Pat. No. 5,394,892; U.S. Pat. No. 4,588,383; U.S. Re. Pat. No. 34,800; U.S. Pat. No. 4,290,114; U.S. Pat. No. 5,521,812; and U.S. Des. Pat. No. 249,548.

While these devices fulfill their respective, particular objectives and requirements, the aforementioned patents do not disclose a new action inducing device. The inventive device includes

a microprocessor adapted for processing received electronic data and sending electronic audio signals. A plurality of storage medium electronically stores data. Each of the storage medium has a different set of instructions thereon. Each of the storage medium provides one set of instructions in a plurality of languages. A reading device reads the electronically stored data on the storage media. The reading device is electrically coupled to the microprocessor. A is electrically coupled to the microprocessor. A plurality of actuators is used for selecting one of the languages. The actuators are operationally coupled to the microprocessor.

In these respects, the action inducing device according to the present invention substantially departs from the conventional concepts and designs of the prior art, and in so doing provides an apparatus primarily developed for the purpose of giving instructions to complete various tasks in a plurality of languages.

### SUMMARY OF THE INVENTION

In view of the foregoing disadvantages inherent in the known types of action inducing devices now present in the prior art, the present invention provides a new action inducing device construction wherein the same can be utilized for giving instructions to complete various tasks in a plurality of languages.

The general purpose of the present invention, which will be described subsequently in greater detail, is to provide a new action inducing device apparatus and method which has many of the advantages of the action inducing devices mentioned heretofore and many novel features that result in a new action inducing device which is not anticipated, rendered obvious, suggested, or even implied by any of the prior art action inducing devices, either alone or in any combination thereof.

To attain this, the present invention generally comprises a microprocessor adapted for processing received electronic data and sending electronic audio signals. A plurality of storage medium electronically stores data. Each of the storage medium has a different set of instructions thereon. Each of the storage medium provides one set of instructions in a plurality of languages. A reading device reads the electronically stored data on the storage media. The reading device is electrically coupled to the microprocessor. A is electrically coupled to the microprocessor. A plurality of actuators is used for selecting one of the languages. The actuators are operationally coupled to the microprocessor.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

It is therefore an object of the present invention to provide a new action inducing device apparatus and method which has many of the advantages of the action inducing devices mentioned heretofore and many novel features that result in a new action inducing device which is not anticipated, rendered obvious, suggested, or even implied by any of the prior art action inducing devices, either alone or in any combination thereof.

It is another object of the present invention to provide a new action inducing device which may be easily and efficiently manufactured and marketed.

It is a further object of the present invention to provide a new action inducing device which is of a durable and reliable construction.

An even further object of the present invention is to provide a new action inducing device which is susceptible of a low cost of manufacture with regard to both materials and labor, and which accordingly is then susceptible of low prices of sale to the consuming public, thereby making such action inducing device economically available to the buying public.

Still yet another object of the present invention is to provide a new action inducing device which provides in the apparatuses and methods of the prior art some of the advantages thereof, while simultaneously overcoming some of the disadvantages normally associated therewith.

Still another object of the present invention is to provide a new action inducing device for giving instructions to complete various tasks in a plurality of languages.

Yet another object of the present invention is to provide a new action inducing device which includes a microprocessor adapted for processing received electronic data and sending electronic audio signals. A plurality of storage medium electronically stores data. Each of the storage medium has a different set of instructions thereon. Each of the storage medium provides one set of instructions in a plurality of languages. A reading device reads the electronically stored data on the storage media. The reading device is electrically coupled to the microprocessor. A is electrically coupled to the microprocessor. A plurality of actuators is used for selecting one of the languages. The actuators are operationally coupled to the microprocessor.

Still yet another object of the present invention is to provide a new action inducing device that allows a person to have one device for offering rescuing information in a plurality of languages. This device has great importance in household setting where servants may not speak the same language as the employer. The storage medium may be programmed with a plurality of languages.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be made to the accompanying drawings and descriptive matter in which there are illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a schematic perspective view of a new action inducing device according to the present invention.

FIG. 2 is a schematic plan view of a placard of the present invention.

FIG. 3 is a schematic perspective view of the present invention.

FIG. 4A is a schematic flow chart representative of the steps of performing CPR.

FIG. 4B is a schematic flow chart representative of the steps of performing CPR.

FIG. 5 is an electronic schematic of the present invention.

### DESCRIPTION OF THE PREFERRED EMBODIMENT

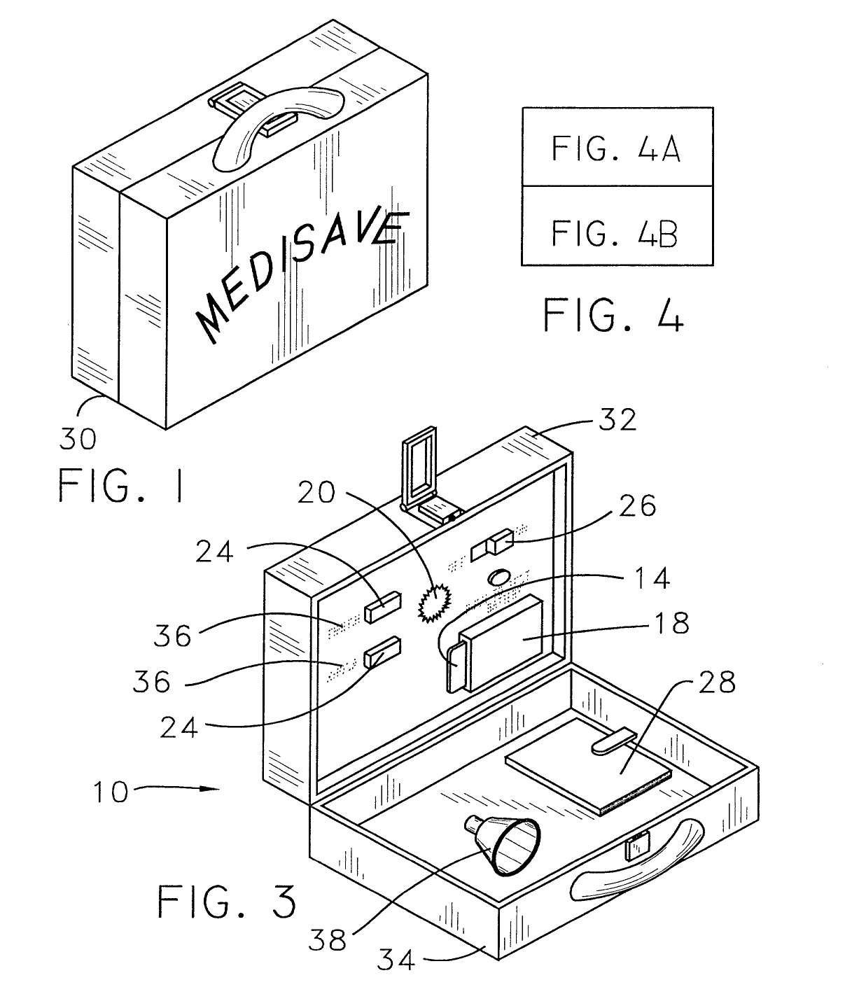

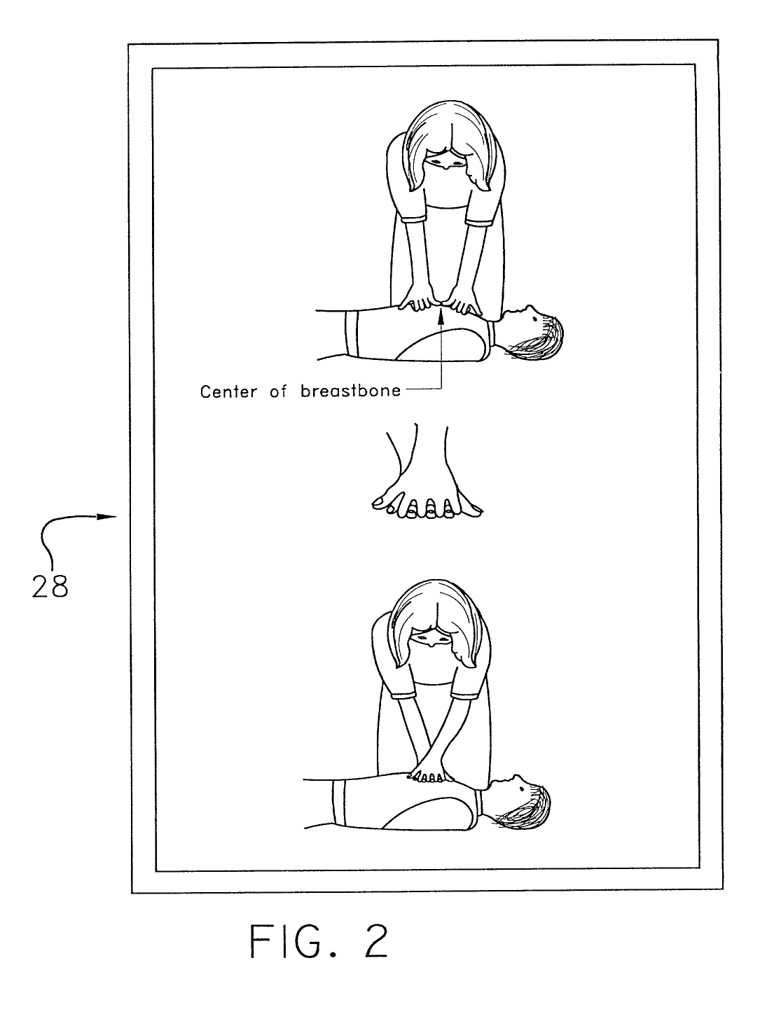

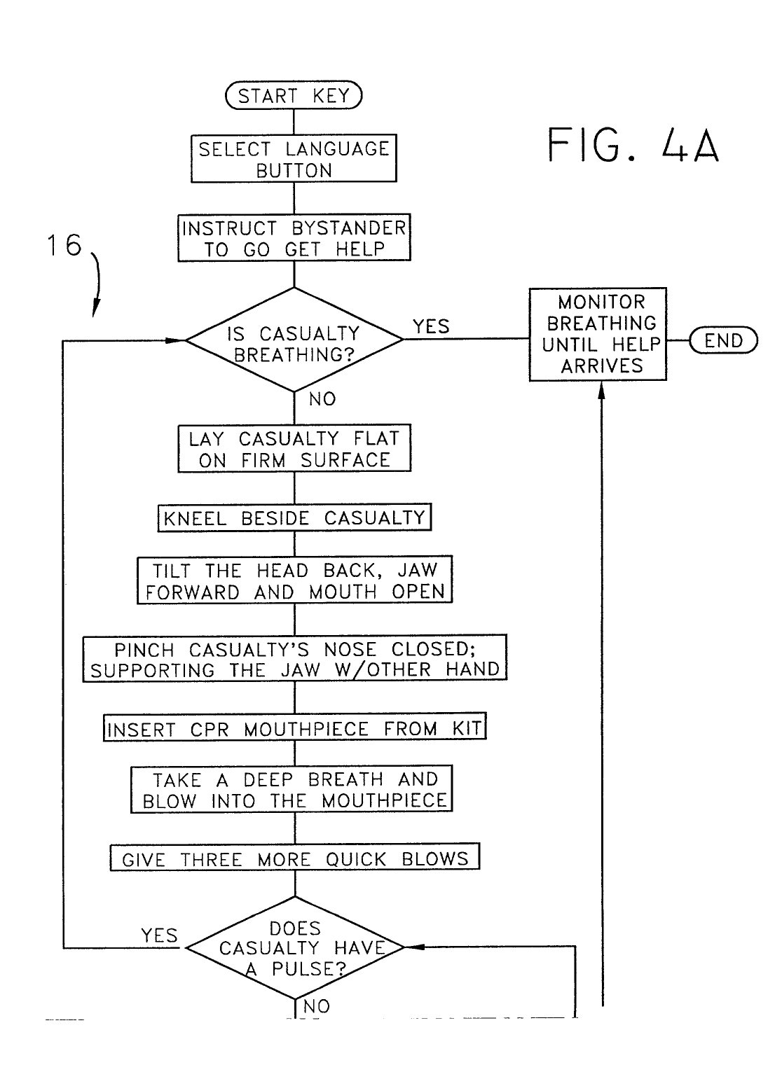

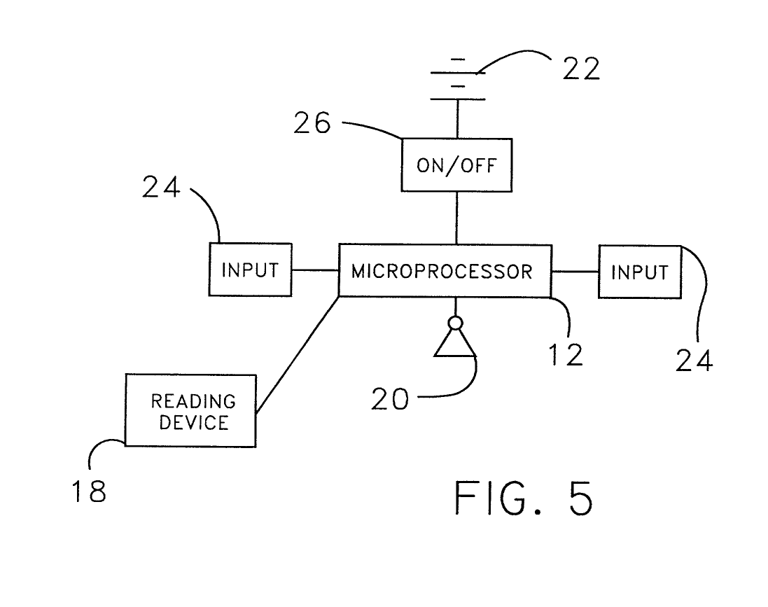

With reference now to the drawings, and in particular to FIGS. 1 through 5 thereof, a new action inducing device embodying the principles and concepts of the present invention and generally designated by the reference numeral 10 will be described.

As best illustrated in FIGS. 1 through 5, the action inducing device 10 generally includes a microprocessor 12 adapted for processing received electronic data and sending electronic audio signals.

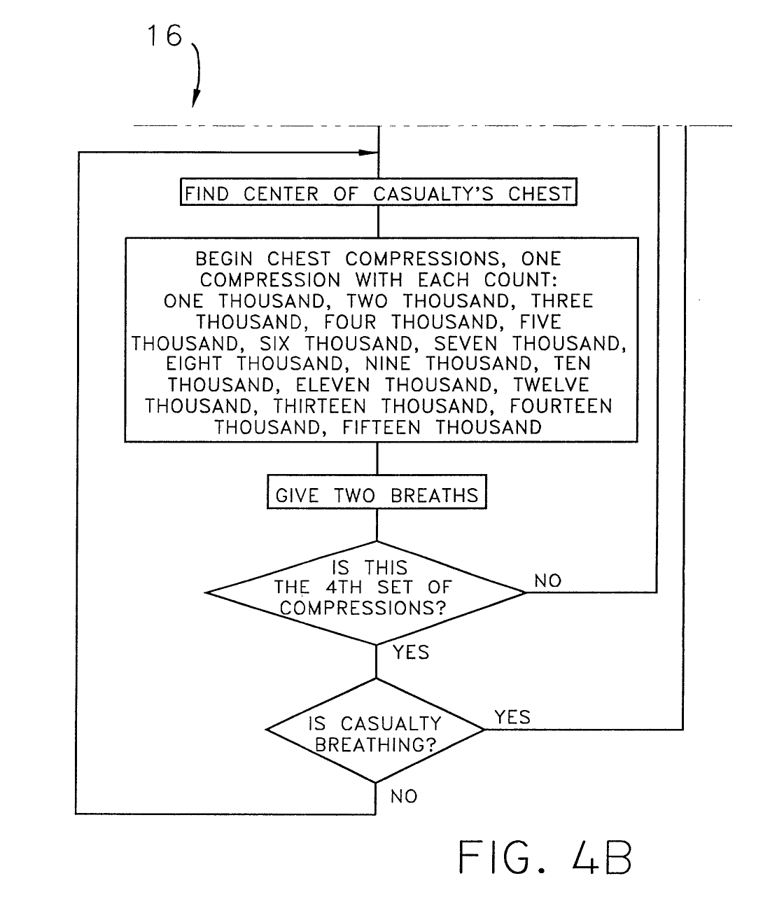

A plurality of storage medium 14 electronically stores data for processing by the microprocessor 12. Each of the storage medium 14 has a different set of instructions thereon. Each of the sets of instructions is selected from the group consisting of performing cardiopulmonary resuscitation (CPR) 16, resuscitating a drowning victim, changing a tire, and reviving an electrocution victim. Each of the storage medium 14 comprises a card having a magnetic strip securely attached thereto for storing programmed data thereon. Alternatively, the card may contain an electronically programmed ROM (EPROM) chip, or a static RAM chip which may pre-programmed with the required data. Each of the storage medium 14 provides one set of instructions in a plurality of languages, such as French, English, Spanish, German, Japanese and Chinese. FIGS. 4A and 4B are representative of instructions given for performing CPR.

A reading device 18 for reading electronically stored data is electrically coupled to the microprocessor 12. The reading device 18 is adapted for receiving one of the cards and reading information on an associated magnetic strip, EPROM chip or ROM chip.

A speaker 20 for emitting audible sounds is electrically coupled to the microprocessor.

A power supply 22 for powering the microprocessor is electrically coupled to the microprocessor 12. The power supply 22 is preferably a rechargeable battery.

A plurality of actuators 24 is provided for selecting one of the plurality of languages stored on the storage medium 14. Each of the actuators 24 indicates one of the languages stored on the storage medium. Each of the actuators 24 is operationally coupled to the microprocessor 12.

A switch 26 selectively turns the microprocessor on and off. The switch 26 is operationally coupled to the microprocessor 12.

A plurality placards 28 visually indicate the instructions. Each of the placards 28 has indicia thereon indicating one of the set of instructions.

A case 30 has a top portion 32 hingedly coupled to a bottom portion 34. The top portion 32 has the microprocessor 12, the speaker 20, the actuators 24, the switch 26 and the reading device 36 securely 18 attached thereto. There are preferably written indicia next to the actuators 32 indicating the language to be chosen. The bottom portion 34 defines a container for holding the placards 28.

Ideally, a conventional cardiopulmonary resuscitation mouthpiece 38 is positioned in the case.

In use, the person opens the case and turns the microprocessor on. The storage medium is positioned in the reading device and the actuator corresponding to the language desired is pressed to give instructions in that language. The speaker emits audible instructions for performing the desired task.

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. An action prompting device for prompting a user's actions during an emergency situation, said device comprising:
a microprocessor adapted for processing received electronic data and sending electronic audio signals;
a plurality of storage medium for electronically storing data, each of said storage medium having a different set of instructions thereon, each of said storage medium providing one set of instructions in a plurality of languages;
a reading device for reading electronically stored data on said storage media, said reading device being electrically coupled to said microprocessor;
a speaker for emitting audible sounds, said speaker being electrically coupled to said microprocessor; and
a plurality of actuators for selecting one of said plurality of languages, each of said actuators indicating one of said languages, each of said actuators being operationally coupled to said microprocessor.

2. The action prompting device as in claim 1, wherein each of said sets of instructions is different instructions selected from the group consisting of performing cardiopulmonary resuscitation, resuscitating a drowning victim, changing a tire, and reviving an electrocution victim.

3. The action prompting device as in claim 1, further including:
a switch for selectively turning said microprocessor on and off, said switch being operationally coupled to said microprocessor.

4. The action prompting device as in claim 1, further including:
a plurality of placards for visually indicating said instructions, each of said placards having indicia thereon indicating one of said set of instructions.

5. The action prompting device as in claim 4, further including:
a case, said case having a top portion hingedly coupled to a bottom portion, said top portion having said microprocessor, said speaker, said actuators, said switch and said reading device securely attached thereto, said bottom portion defining a container for holding said placards.

6. The action prompting device as in claim 1, further including:
a case, said case having a top portion hingedly coupled to a bottom portion, said top portion having said microprocessor, said speaker, said actuators, said switch and said reading device securely attached thereto.

7. The action prompting device as in claim 5, further including:
a cardiopulmonary resuscitation mouthpiece being positioned in said case.

8. An action prompting device for prompting a user's actions during an emergency situation, said device comprising:
a microprocessor adapted for processing received electronic data and sending electronic audio signals;
a plurality of storage medium for electronically storing data, said storage medium having a set of instructions thereon, said set of instructions including performing cardiopulmonary resuscitation, said set of instructions being provided in at least two languages on said storage medium;
a reading device for reading electronically stored data on said storage medium;
a speaker for emitting audible sounds, said speaker being electrically coupled to said microprocessor;
a power supply for powering said microprocessor, said power supply being electrically coupled to said microprocessor;
a plurality of actuators for selecting one of said plurality of languages, each of said actuators indicating one of said languages, each of said actuators being operationally coupled to said microprocessor;
a switch for selectively turning said microprocessor on and off, said switch being operationally coupled to said microprocessor;
a case, said case having a top portion hingedly coupled to a bottom portion, said top portion having said microprocessor, said speaker, said actuators, said switch and said reading device securely attached thereto; and
a cardiopulmonary resuscitation mouthpiece being positioned in said case.

9. An action prompting device for prompting a user's actions during an emergency situation, said device comprising:
a microprocessor adapted for processing received electronic data and sending electronic audio signals;
a plurality of storage medium for electronically storing data, each of said storage medium having a different set of instructions thereon, each of said sets of instructions being different instructions selected from the group consisting of performing cardiopulmonary resuscitation, resuscitating a drowning victim, changing a tire, and reviving an electrocution victim, each of said storage medium comprising a card having a magnetic strip securely attached thereto and for storing said data thereon, each of said storage medium providing one set of instructions in a plurality of languages;
a reading device for reading electronically stored data, said reading device being electrically coupled to said microprocessor, said reading device being adapted for receiving one of said cards and reading information on an associated magnetic strip;
a speaker for emitting audible sounds, said speaker being electrically coupled to said microprocessor;
a power supply for powering said microprocessor, said power supply being electrically coupled to said microprocessor;
a plurality of actuators for selecting one of said plurality of languages, each of said actuators indicating one of said languages, each of said actuators being operationally coupled to said microprocessor;
a switch for selectively turning said microprocessor on and off, said switch being operationally coupled to said microprocessor;
a plurality of placards for visually indicating said instructions, each of said placards having indicia thereon indicating one of said set of instructions;
a case, said case having a top portion hingedly coupled to a bottom portion, said top portion having said microprocessor, said speaker, said actuators, said switch and said reading device securely attached thereto, said bottom portion defining a container for holding said placards; and
a cardiopulmonary resuscitation mouthpiece being positioned in said case.
